(19) United States
(12) Patent Application Publication
(10) Pub. No.: US 2015/0133713 A1
(43) Pub. Date: May 14, 2015

(54) Method For Compensating The Deviation Of A Hadron Beam Produced By A Hadron-Therapy Installation

(71) Applicant: Ion Beam Applications S.A., Louvain-La-Neuve (BE)

(72) Inventors: Olivier Vierlinck, Liernu (BE); Michel Closset, Avin (BE); David Wikler, Louvain-La-Neuve (BE)

(21) Appl. No.: 14/399,477

(22) PCT Filed: May 10, 2013
(86) PCT No.: PCT/EP2013/059726

§ 371 Date: Nov. 6, 2014

(30) Foreign Application Priority Data
May 9, 2012 (EP) 12167395.8
May 14, 2012 (EP) 12167902.1

(51) Int. Cl. A61N 5/10

(52) U.S. Cl. CPC A61N 5/1067; A61N 5/1081; A61N 5/1045
USPC 600/1

(57) ABSTRACT

The present invention relates to a method for compensating the deviation of an energetic beam of hadrons delivered by an irradiation unit of a hadron-therapy installation, with respect to an isocentre of a rotatable gantry supporting said irradiation unit, said irradiation unit comprising a collimator comprising an opening for the passage of said beam. The present invention also relates to a program comprising an algorithm for calculation of a correction to be applied to the position of an opening of a collimator of an irradiation unit of a hadron-therapy installation. The invention finally also relates to a hadron therapy installation.

### TECHNICAL FIELD

According to a first aspect, the present invention relates to a method for compensating the deviation of a beam of hadrons with respect to an isocentre, said beam of hadrons being produced by a hadron-therapy installation comprising a rotatable gantry equipped with an irradiation unit. According to a second aspect, the present invention relates to a program allowing this deviation to be compensated.

### DESCRIPTION OF THE PRIOR ART

The recent techniques of hadron-therapy for the treatment of cancers allow a dose to be delivered with precision onto a target volume, for example a tumour, while at the same time preserving the surrounding tissues. A hadron-therapy installation comprises a particle accelerator producing a beam of charged particles, a rotatable gantry comprising a means of transport for the beam and an irradiation unit. The irradiation unit delivers a dose distribution onto the target volume and generally comprises means for controlling the dose delivered, such as for example an ionization chamber, together with means for controlling the direction or the shape of the beam.

Two major modes of delivering beams are employed in hadron-therapy: a first delivery mode comprises the techniques known as passive scattering of the beam, and a second, more elaborate, mode of treatment comprises the dynamic beam scanning techniques.

The techniques of passive scattering of the beam make use of an assembly of elements allowing the path of the particles to be adjusted up to the point of maximum depth of the region to be irradiated and over the required width, and in order to obtain a dose that coincides as far as possible with the target volume, it is necessary to use accessories such as a compensator and/or a collimator specific to the tumour of the patient and to the angle of irradiation. These accessories are generally disposed on a telescopic device situated at the end of the irradiation unit. The telescopic device allows the accessories to be brought together near to the area of the patient to be treated.

In the dynamic beam scanning techniques, such as for example the PBS (Pencil Beam Scanning) technique, described in the document EP1147693, a fine beam of particles oriented along a z axis is scanned in a plane orthogonal to this z axis over the target volume by means of scanning magnets. By varying the energy of the beam of particles, various layers within the target volume may be successively irradiated. In this way, the dose of radiation can be delivered over the entirety of the target volume. The PBS techniques allow a target volume to be irradiated without having to resort to either a collimator or an energy compensator specific to the patient. Nevertheless, in certain cases, the use of a collimator is however preferred.

The majority of the hadron-therapy installations comprise a treatment room with a rotatable gantry supporting a system for delivering the beam comprising a beam transport line at the end of which an irradiation unit is positioned. The rotatable gantry is able to rotate about a horizontal axis of rotation, in such a manner that the irradiation unit can deliver a treatment beam along several angles of irradiation.

A point in space is defined at the intersection between the horizontal axis of rotation of the gantry and the central axis of the beam delivered by the irradiation unit along a plurality of angles of rotation of the gantry. In practice, all the central axes of beams delivered according to several angles of rotation of the gantry do not cross one another at a single point but each intercept a small spherical or ellipsoidal volume. The centre of mass of this spherical or ellipsoidal volume is generally defined as the isocentre of the rotatable gantry. In other words, the so defined isocentre is a fixed isocentre, being a single reference point for a gantry of the hadron therapy installation. The central axis of a beam when delivered at different gantry angles does, as mentioned above, generally not pass through this so defined fixed isocentre. In practice one can measure the deviation of the beam trajectory from this defined fixed isocentre. Those deviations of a fixed isocentre are determined for example by the method described in the document U.S. Pat. No. 7,349,523 or the method described in the document EP2186542. When the word isocentre is further used in the current document, it is to be understood as a single point defined in a gantry treatment room of the hadron therapy facility.

The treatment beam is delivered according to the specifications of a treatment plan. The patient is positioned in a precise manner such that the area to be treated is irradiated in accordance with the target volume specified by the treatment plan. The treatment plan is based on data related to the position and the shape of the area to be treated obtained by imaging of the patient.

In order to minimize the dose on the healthy tissues between the region of entry of the beam into the patient and the area to be treated, the patient is positioned in a precise manner such that the area to be treated is centred on the isocentre.

As explained hereinabove, all the central axes of beams delivered according to several angles of rotation of the gantry do not cross at a single point but each intercept a small spherical or ellipsoidal volume. Depending on the angle of irradiation, the central axis of the beam can either go through the isocentre or deviate with respect to the position of the isocentre. Other parameters can have an influence on the deviation of the beam with respect to the position of the isocentre, such as for example:
the extension of the telescopic support supporting the accessories;
the weight of the accessories;
the type of telescopic device used and adapted to the weight of the accessories;
the mode of treatment (double scattering, Uniform scanning, Pencil beam scanning or Single scattering);
the irradiation parameters (position, presence of beam modulator, of beam widener, etc.).

The angle of rotation of the gantry and the extension of the telescopic support are generally considered as having a significant influence on the deviation of the beam with respect to the position of the isocentre. The significant influence of these parameters on the deviation of the beam is explained by:
bending of the irradiation unit under its own weight, dependent on the angle adopted by the rotatable gantry;
a limited mechanical rigidity of the rotatable gantry and of its support structure;
bending of the telescopic device varying according to the extension of this telescopic device.

In order to compensate these deviations of the beam with respect to the position of the isocentre as a function of the treatment angle, a correction to be applied to the position of the patient is generally calculated and the patient is re-positioned for each new treatment angle. Each re-positioning of the patient involves a loss of time and requires a verification of the alignment of the patient with respect to the beam. An example of this type of positioning system and method is given in document EP1759733. This verification generally comprises a step of X-ray imaging for the positioning of the area to be treated, a process which leads to the patient being subjected to additional doses of the beam.

A hadron-therapy installation can also comprise a treatment room that is not equipped with a rotatable gantry but with a so-called fixed beam line. In such systems the irradiation unit is fixed on a support that is attached to the floor. For those fixed beam rooms, a reference point in the treatment room where the target to be irradiated has to be positioned, is defined as well. This reference point is sometimes also called an isocentre although there is only one beam direction. This reference point or isocentre in a fixed beam treatment room can for example be defined as a point located on what is defined as the central beam direction of the beam delivered by the fixed irradiation unit and located at a well defined distance from the fixed irradiation unit. As in the gantry treatment room, the irradiation unit in a fixed beam treatment room may be equipped with accessories and/or telescopic positioning devices. For a fixed beam treatment room, a deviation of the beam from the reference point or isocentre may be caused by the any of the following parameters:
extension of the telescopic support supporting the accessories;
the weight of the accessories;
the type of telescopic device used and adapted to the weight of the accessories;
the mode of treatment (double scattering, Uniform scanning, Pencil beam scanning or Single scattering);
the irradiation parameters (position, presence of beam modulator, of beam widener, etc.)

Both for a gantry treatment room and for a fixed beam treatment room, it would be desirable to minimize or to eliminate the effects of the deviation of the beam with respect to the position of the isocentre so as to no longer need to re-position the patient for each treatment angle.

### SUMMARY OF THE INVENTION

The invention is related to a method and to a program and installation as disclosed in the appended claims. Everywhere in this description, the word ‘program’ preferably refers to a ‘computer program’.

The invention is thus related firstly to a method for compensating the deviation of an energetic beam of hadrons delivered by an irradiation unit of a hadron-therapy installation, with respect to an isocentre of a structure supporting said irradiation unit, said irradiation unit being configured for receiving a collimator having an opening for the passage of said beam, said compensation method comprising the steps of:
i. determining the deviation of the beam with respect to said isocentre as a function of a parameter of said installation that can have an influence on the deviation of said beam, thereby obtaining a calibration curve of said deviation as a function of a plurality of values of said parameter;
ii. obtaining a treatment plan from a treatment planning system, said plan defining a prescribed shape and position of said collimator opening for performing a treatment,
iii. calculation of a correction to be applied to said prescribed position of said opening for compensating the deviation of said beam with respect to said isocentre, said correction being calculated on the basis of said calibration curve;
iv. application of said correction to said prescribed position of said opening.

The invention is also related to a program comprising an algorithm for calculation of a correction to be applied to the position of an opening of a collimator of an irradiation unit of a hadron-therapy installation comprising a structure supporting said irradiation unit, the shape and the position of said opening being prescribed by a treatment plan, said correction to be applied being capable of compensating the deviation of the beam produced by said hadron-therapy installation, with respect to the isocentre of said structure, as a function of a parameter that can have an influence on the direction of the beam, wherein said correction is calculated on the basis of a calibration curve of said deviation as a function of a plurality of values of said parameter.

The treatment plan referred to in the two preceding paragraphs is specific to a patient and to the target within the patient that needs a treatment. No positioning of the patient is needed for compensating the deviation. In the method and program of the invention, the isocentre is defined as a point that has a fixed position with respect to coordinate system that is not attached to the irradiation unit, for example a point with a fixed position with respect to the supporting structure itself or with respect to the space wherein said structure is located.

According to different embodiments, the structure can be a rotatable gantry or a fixed beam structure. Further specific embodiments are set out hereafter and in the appended claims.

As stated, the correction is calculated on the basis of the calibration curve. This means that a value of said parameter is applied and the corresponding deviation is read from the curve, after which the correction is calculated. The value to be applied for the parameter may for example be obtained from the treatment plan (e.g. prescribed gantry rotation angle) or from any other source (e.g. weight of the accessories).

The invention is equally related to a hadron-therapy installation for delivering a hadron beam for hadron therapy, said hadron-therapy installation comprising
an irradiation unit for delivering said beam, said irradiation unit being configured for receiving a collimator having an opening for the passage of the beam,
a structure for supporting said irradiation unit,
a treatment planning system for providing a treatment plan, said treatment plan comprising prescribed collimator data defining the shape of the opening and the position of the opening of the collimator, ps characterized in that
   said hadron-therapy installation further comprises
a storage medium configured for storing data related to the deviation of the beam with respect to an isocentre as a function of one or more irradiation parameters of said installation that can have an influence on the deviation of said beam,
   a plan modifier controller adapted to modify said prescribed collimator data of said treatment plan by defining a correction to the position of said opening of said collimator, wherein said defining a correction is based on said data related to the deviation of the beam.

The present invention relates to a method for compensating the deviation of an energetic beam of hadrons delivered by an irradiation unit of a hadron-therapy installation, with respect to an isocentre of a rotatable gantry supporting said irradiation unit, said irradiation unit comprising a collimator comprising an opening for the passage of said beam, said compensation method comprising the steps for:
i) determining the deviation of the beam with respect to said isocentre as a function of a parameter of said installation that can have an influence on the deviation of said beam;
ii) calculation of a correction to be applied to the position of said opening for compensating the deviation of said beam with respect to said isocentre;
iii) application of said correction to the position of said opening.

Preferably, the parameter that can have an influence on the deviation of said beam is the angle of rotation of said rotatable gantry.

Preferably, the irradiation unit comprises a telescopic support for the positioning of accessories such as said collimator associated or not with a compensator, the method being characterized in that the deviation of the beam with respect to said isocentre is determined as a function of at least a second parameter that can have an influence on the deviation of said beam selected from amongst:
the extension of the telescopic support supporting the accessories;
the weight of the accessories;
the type of telescopic support used and adapted to the weight of the accessories;
the mode of treatment (double scattering, Uniform scanning, Pencil beam scanning or Single scattering);
the irradiation parameters (position, presence of beam modulator, of beam widener, etc.).

Preferably, said step for determining the deviation of the beam with respect to said isocentre as a function of a parameter is carried out according to the following sub-steps:
a) irradiation of a detector using a calibration collimator, said detector being positioned with respect to the isocentre;
b) measurement of the beam field by means of said detector;
c) determination of the centre of the beam field;
d) measurement of the offset between the centre of the measured beam field and the isocentre;
e) change of one or more of said parameters that can have an influence on the deviation of the beam and repetition of the steps a) to d).

Preferably, when said collimator is a multi-leaf collimator, said step for application of said correction to the position of said opening comprises a step for displacement of said leaves.

Preferably, when said collimator is a block comprising an opening, and when said collimator is fixed onto a device capable of moving it in a plane perpendicular to the direction of the beam, said step for application of said correction to said opening comprises a step for displacement of said collimator by means of said device.

Preferably, said step for application of said correction to said opening comprises a step for fabrication of a collimator whose opening shape and position is based on said treatment plan and on the calculation of said correction to be applied.

Preferably, said collimator is associated with a compensator comprising a hollow part whose shape is predetermined on the basis of said treatment plan and aligned with said opening of said collimator.

The present invention also relates to a program comprising an algorithm for calculation of a correction to be applied to the position of an opening of a collimator of an irradiation unit of a hadron-therapy installation comprising a rotatable gantry supporting said irradiation unit, the shape and the position of said opening being defined by a treatment plan, said correction to be applied being capable of compensating the deviation of the beam produced by said hadron-therapy installation, with respect to the isocentre of said gantry, as a function of a parameter that can have an influence on the direction of the beam.

Preferably, said parameter that can have an influence on the direction of said beam is the angle of rotation of said rotatable gantry.

Preferably, said irradiation unit comprises a telescopic support for the positioning of accessories such as said collimator associated or not with a compensator, the program being characterized in that said correction to be applied is capable of compensating the deviation of the beam produced by said hadron-therapy installation with respect to the isocentre of said gantry, as a function of one or more second parameter(s) selected from amongst:
the extension of the telescopic support 111 supporting the accessories 108, 110;
the weight of the accessories 108, 110;
the type of telescopic support used and adapted to the weight of the accessories;
the mode of treatment (double scattering, Uniform scanning, Pencil beam scanning or Single scattering);
the irradiation parameters (position, presence of beam modulator, of beam widener, etc.).

Preferably, the program is capable of establishing a calibration curve showing the deviation of the beam with respect to the isocentre as a function of one or more of said parameters that can have an influence on the deviation of the beam.

Preferably, the program creates corrected position data for the opening of a collimator,

on the basis of data coming from a treatment plan defining the shape and the position of said opening in the collimator for a given isocentre; and
on the basis of the calculation of the correction to be applied to the position of said opening.

Preferably, said corrected position data for the opening of the collimator are transmitted to a device capable of forming said opening.

Preferably, said device capable of forming said opening is a control system for motorization of a plurality of leaves of a multi-leaf collimator.

Preferably, said device capable of forming said opening is a control system for a collimator fabrication device.

Preferably, said corrected position data for the opening of said collimator are transmitted to a printer device for printing a plan of said collimator on a 1:1 scale, said plan being designed to verify the correct position of said collimator.

Preferably, said corrected position data are transmitted to a device capable of moving said collimator.

### BRIEF DESCRIPTION OF THE FIGURES

FIG. 1 shows a hadron-therapy installation.

FIG. 2a shows an irradiation unit of a hadron-therapy installation, the irradiation unit being equipped with a collimator formed from a single block.

FIG. 2b shows an irradiation unit of a hadron-therapy installation, the irradiation unit being equipped with a collimator formed from a single block and with a compensator.

FIG. 3 shows an irradiation unit of a hadron-therapy installation, the irradiation unit being equipped with a multi-leaf collimator.

FIG. 4 shows an irradiation unit of a hadron-therapy installation, the irradiation unit being equipped with a collimator formed from a single block and with a device for moving said collimator in a plane perpendicular to the axis of the beam.

FIGS. 5a and 5b show one example of a calibration collimator 108′ for which a simple shape of opening 109′ has been defined, as shown in FIG. 5a, said calibration collimator being able to be moved in a plane perpendicular to the axis of the beam by means of a device 113.

FIG. 6 shows a flow diagram presenting the steps of the method of the present invention.

FIG. 7a shows an irradiation unit delivering a beam at three different gantry angles according to the prior art.

FIG. 7b shows an irradiation unit delivering a beam at three different gantry angles according to the invention.

FIG. 8 illustrates a method according to the invention.

### DESCRIPTION OF THE INVENTION

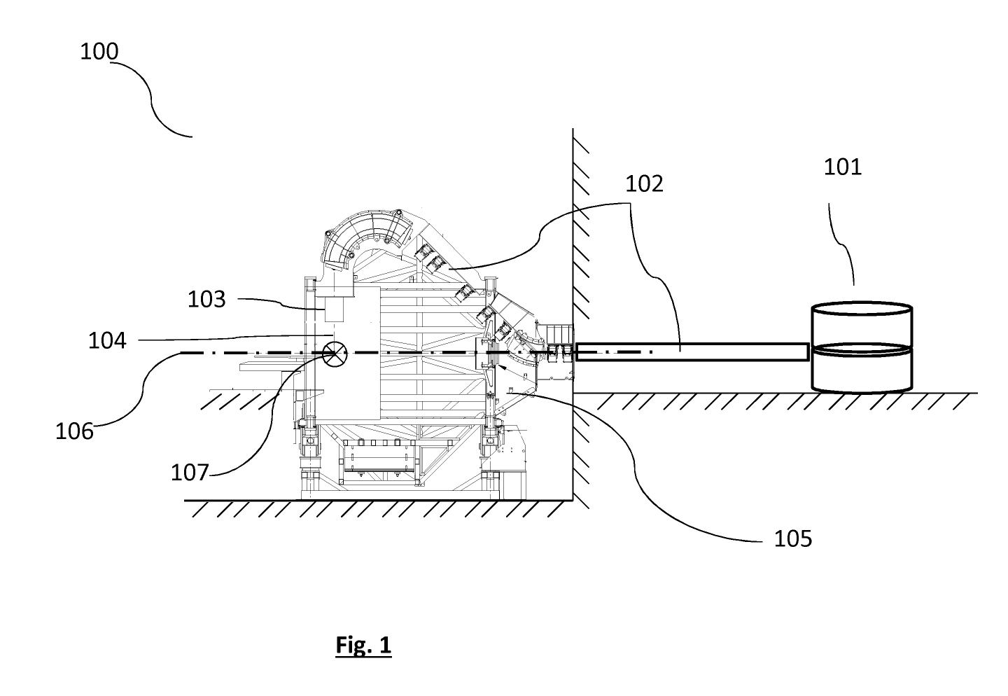

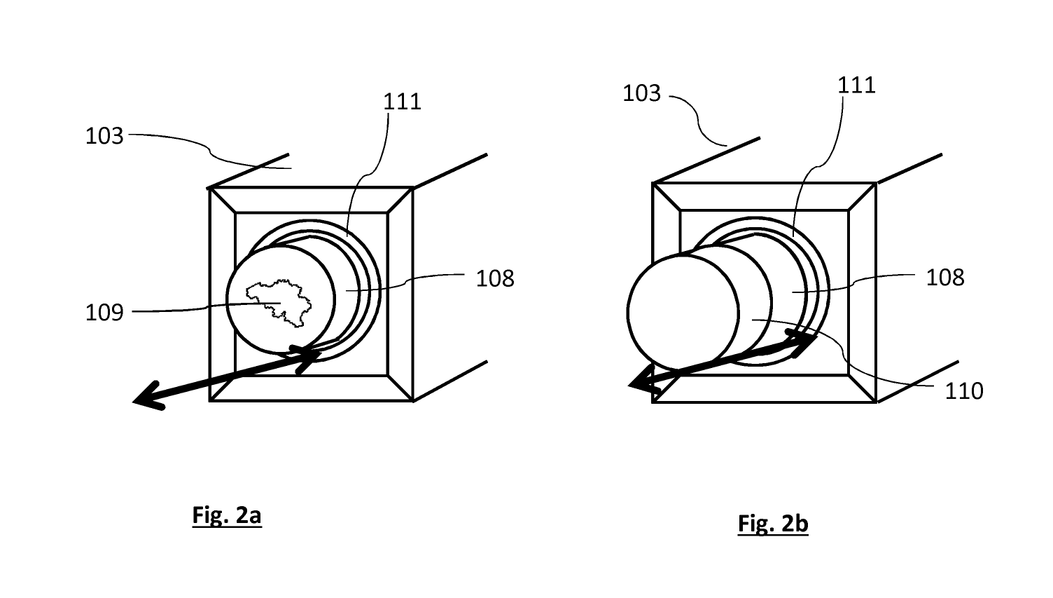

FIG. 1 shows a hadron-therapy installation 100 comprising:
an accelerator 101 capable of producing an energetic beam of hadrons;
a beam transport line 102 at the end of which an irradiation unit 103 is positioned, this irradiation unit comprising one or more accessories 108, 110 shown in more detail in FIGS. 2 to 4;
a rotatable gantry 105 partially supporting said beam transport line and capable of rotating about a horizontal axis of rotation 106.

The representation of the hadron-therapy installation in this FIG. 1 is not limiting for the present invention. The invention is applicable to other models of hadron-therapy installation, such as for example an installation comprising a compact rotatable gantry such as described in the document EP2308561 or a hadron-therapy installation in which the rotatable gantry supports the entirety of the beam transport line, or again an installation in which the rotatable gantry supports both the beam transport line and the accelerator, as is described in the document WO2012014715. Finally, the invention is also related to a hadron-therapy installation comprising a fixed beam structure, as will be described further.

FIGS. 2a, 2b, 3 and 4 show various models of irradiation unit 103 comprising a collimator 108. The collimator 108 is generally made of brass and comprises an opening 109 whose shape and position are predetermined according to a treatment plan obtained by a treatment planning system, in such a manner as to tailor the dose of the beam in the lateral plane so that it coincides as far as possible with the shape of the target volume. In other words, the opening of the collimator is generally made patient specific. In radiotherapy, a collimator is sometimes also named aperture or block. For the passive techniques of delivery of the beam, the collimator 108 is often associated with a range compensator 110. The range compensator 110 is generally made of Plexiglas (methylpolymethacrylate) and comprises a hollow portion with a variable thickness aligned with said opening, facing said opening 109. The shape of the compensator is also predetermined by the treatment plan system in such a manner as to adjust the dose depth distribution. For the PBS delivery technique, a collimator, either a patient-specific collimator or a non patient-specific collimator can also be used. If the collimator is not patient specific, the opening of the collimator can have for example a geometrical shape such as a rectangle or a circular shape.

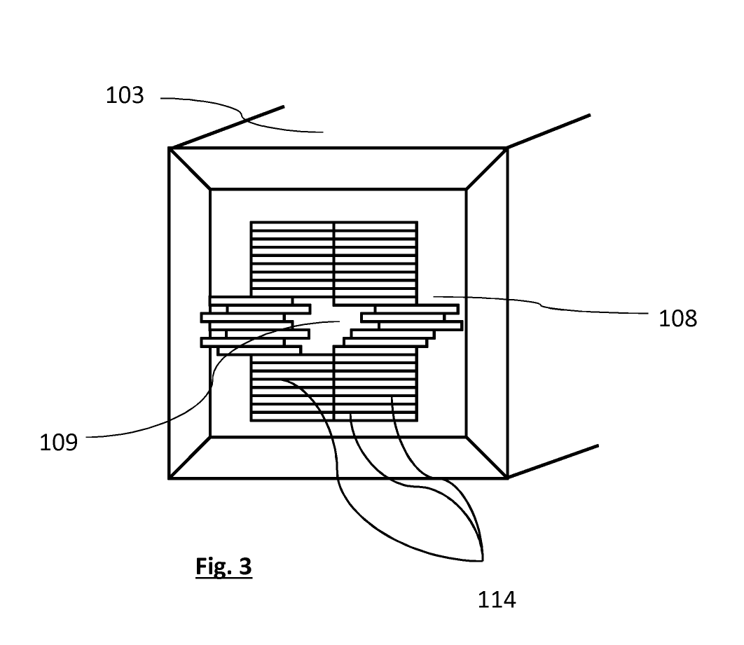

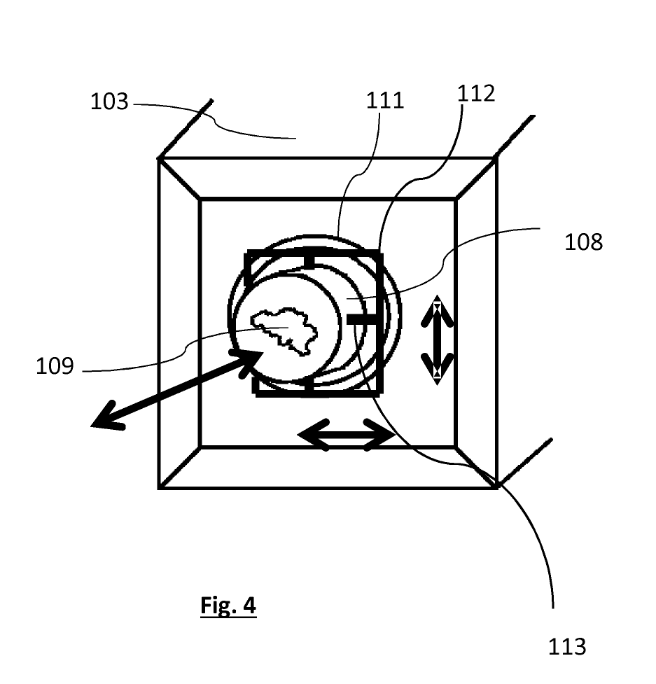

Several types of collimators exist that are used in hadron-therapy:
multi-leaf collimators such as shown in FIG. 3 comprising a plurality of leaves 114 disposed parallel to and against one another that can be actuated via a motorization device (not shown) so as to form a predefined opening 109; and
collimators formed from a single block such as shown in FIGS. 2a, 2b and 4, generally made of brass and comprising a predefined opening 109.

The treatment planning system generally provides prescribed data that define the prescribed opening in the collimator that is needed to perform a specific treatment. Based on these prescribed data a collimator is generated. A collimator can for example be generated by starting from a plane collimator and then milling the prescribed opening in the collimator based on the prescribed data received from the treatment planning system. Those prescribed data comprise in this case information related to the shape of the opening and the position of the opening on the collimator. In the case of a multi-leaf collimator, the treatment planning system furthermore provides prescribed data defining the prescribed opening of the collimator. However, in this case the data comprise information for setting the positions of the leaves.

In the case of the collimators formed from a single block, the collimator 108 is preferably mounted on a telescopic support 111 sometimes referred to as ‘telescopic nose’, the telescopic support 111, when deployed, allowing the opening 109 of the collimator 108 to be brought as close as possible to the area to be irradiated, and when the telescopic support is retracted, it allows the rotation of the rotatable gantry 105 around the patient. The telescopic support therefore performs a movement in the direction of the axis of the beam 104. The irradiation unit may also comprise a device capable of moving a collimator in a plane perpendicular to the axis of the beam 104 (one example is shown in FIG. 4).

According to a first aspect, the present invention relates to a method for compensating the deviation of an energetic beam of hadrons delivered by a hadron-therapy installation, with respect to the isocentre 107 of the rotatable gantry 105 of said hadron-therapy installation 100.

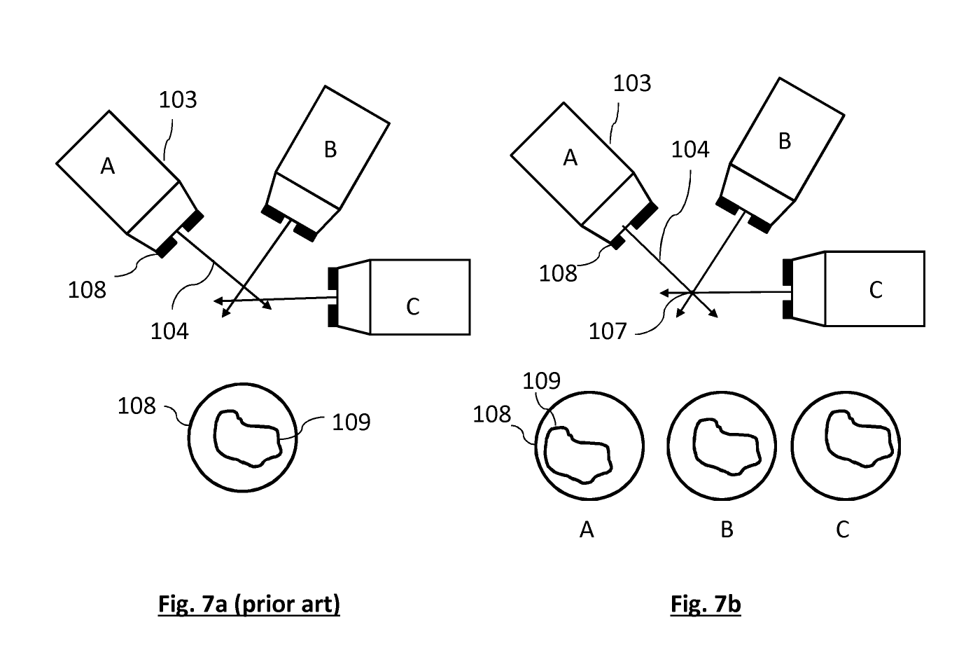

The isocentre 107 of the rotatable gantry 105 is defined, as discussed above, as a fixed reference point located at a well-defined position with respect to the gantry or to the treatment room in which the gantry is placed. In theory, if a gantry would have no deflections, the isocentre of an ideal gantry could for example be defined as the intersection point between the central axis of a beam delivered at different gantry angles. In practice, due to mainly deflections of the gantry and deflections of the irradiation unit, the beams delivered at different gantry angles do not intercept in a single point. This is illustrated in FIG. 7a where an irradiation unit 103 and the central trajectory of the delivered beam 104 is shown for a few different gantry angles A,B,C. As shown on FIG. 7a, the central path of the beams 104 delivered from the different gantry angles do not intercept in a point. As, in practice, no such point exists that could be defined as the single point isocentre for all gantry angles, a theoretical single point isocentre has to be defined. For the purpose of the current invention, the isocentre 107 is to be interpreted as a well defined fixed reference point with respect to a known coordinate system. This reference point can for example be expressed in x,y,z coordinates of a coordinate system corresponding to the coordinate system fixed with respect to the treatment room or fixed with respect to the gantry. The isocentre could for example be defined as the centre of mass of a sphere or of an isocentric ellipsoid or any other volume intercepting each central axis 104′ of a beam delivered by the irradiation unit for each of the angles of rotation of the rotatable gantry. But for the purpose of this invention, any other definition of the isocentre can be used, as long as it is a well defined single reference point with respect to a known coordinate system, preferably defined with respect to the treatment room coordinate system or the gantry coordinate system.

The measurement of deviations of the beam from a fixed isocentre as a function of the gantry angle can be carried out by the methods known from the prior art, such as described in the documents EP2186542 or U.S. Pat. No. 7,349,523.

Several parameters can have an influence on the deviation of the beam with respect to the isocentre:
the angle of rotation of the rotatable gantry;
the extension of the telescopic support 111 supporting the accessories 108, 110;
the weight of the accessories 108, 110;
the type of telescopic support used and adapted to the weight of the accessories;
the mode of treatment (double scattering, Uniform scanning, Pencil beam scanning or Single scattering);
the irradiation parameters (position, presence of beam modulator, of beam widener, etc.).

It is generally considered that the parameters likely to have the greatest influence on the deviation of the beam with respect to the isocentre are:
the angle of rotation of the rotatable gantry and;
the extension of the telescopic support 111 supporting the accessories 108, 110.

With the prior art hadron therapy installations, one generally will correct for those beam deviations by correcting the position of the patient couch as a function of the gantry angle such that for each gantry angle the beam will point to the target to be irradiated. With the method and device of the current invention a conceptually completely different approach is followed in order to correct for those beam deviations. With the method of the invention a single point reference isocentre, preferentially with respect to a treatment room coordinate system or gantry coordinate system, is defined and the deviations of the central beam path with respect to this isocentre are determined as a function of the gantry angle. These deviations are typically determined by performing a calibration measurement prior to the treatment. Such a calibration can be performed a first time when installing the hadron-therapy installation for the first time and then in the framework of a quality assurance program, those deviation measurements can be repeated on a regular basis (e.g. monthly or yearly). Those deviations can for example be measured in steps of one degree of the gantry angle and interpolations can be made for gantry angles located in between the measured gantry angles. In another example, the deviations as a function of gantry angle are only measured at larger intervals, for example every 30°.

The interpolation between the measured deviations leads to a calibration curve of said deviation as a function of a plurality of values of the gantry angle. In other words, said curve allows to determine a beam deviation for a range of values of the gantry rotation angle. According to the invention, for a given treatment at a given treatment gantry angle, the beam deviations at the given treatment angle are corrected by correcting the position of the opening of the collimator based on the deviation obtained from the calibration measurement. For this purpose, the treatment planning data comprising the data defining the position of the opening of the collimator need to be modified accordingly before treatment.

The invention is further illustrated in FIG. 7b. An isocentre 107 is defined as a single point and the prescribed opening of the collimator, as defined by a treatment planning system, is corrected depending on the gantry angle such that when using the corrected openings on the collimator, the central beam trajectories delivered by the irradiation unit at different gantry angles are crossing in a single point. So in this example by using collimators A,B,C with corrected openings for the three gantry angles A,B,C, respectively, the central path of the beams as delivered from the three gantry angles will cross at the isocentre. In the prior art system illustrated in FIG. 7a the opening of the collimator is not corrected with the consequence that when delivering a beam from different angles the beams are not crossing at a single point due to deflections of the gantry. Whereas according to the invention, illustrated in FIG. 7b, the opening of the collimator depends on the gantry angle to be used for the treatment. The correction to be applied to the prescribed opening defined by a treatment planning is based on the measurements of deviations of the beam with respect to a defined reference isocentre 107. Those measurements are performed during a calibration process prior to the treatment. During treatment using openings of the collimator that are corrected based on the calibration beam deviation measurements, beams that are delivered from different gantry angles will point to a single point, being the reference isocentre as illustrated in FIG. 7b.

In general, the correction made to the opening in the collimator comprises a translation with respect to the prescribed opening from the treatment planning system. Such a translation of the prescribed opening is made with respect to the collimator in a plane essentially perpendicular to the direction of the beam. In other words, the contour of the opening remains the same, the contour is just translated. This translation can be expressed as delta-X, delta-Y translation components in an X,Y coordinate system that is fixed with respect to the collimator.

Preferably, said compensation method comprises the steps of:
i. determining the deviation of the beam with respect to said isocentre, as a function of the angle of rotation of the gantry;
ii. calculation of a correction to be applied to said position of said opening such as initially predetermined by the treatment planning system, in order to compensate the deviation of said beam with respect to said isocentre;
iii. application of said correction to said opening.

The calculation of the correction to be applied to a prescribed opening based on the beam deviation calibration measurements can for example be performed based on a coordinate system transformation from the treatment room coordinate system to an irradiation unit coordinate system. Indeed, the deviation of the beam with respect to the isocentre can be measured for example with respect to a treatment room coordinate system. The data of the treatment planning system defining the opening of the collimator is in general defined with respect to a coordinate system that is fixed to the collimator and which is linked with the irradiation unit coordinate system.

Preferably, and in the case of a rotatable gantry supporting the irradiation unit, the compensation method furthermore comprises a step for determining the deviation of the beam with respect to said isocentre as a function of one or more further parameters (in addition to the gantry rotation angle) selected from :
the extension of said telescopic support supporting the accessories;
the weight of the accessories;
the type of telescopic support used and adapted to the weight of the accessories;
the delivery mode of the beam;
the irradiation parameters (position, presence of beam modulator, of beam widener, etc.).
   If the irradiation unit is supported by a fixed beam structure, so that no gantry angle exists, the deviation of the beam with respect to the reference point or isocentre is determined as a function of one or more of these parameters mentioned above.

As stated with respect to the gantry angle, the measurement of the deviation as a function of one or more other parameters defined above leads to one or more further calibration curves of said deviation as a function of a plurality of values of said respective parameters. In other words, said curves allows to determine a beam deviation for a range of values of said parameters.

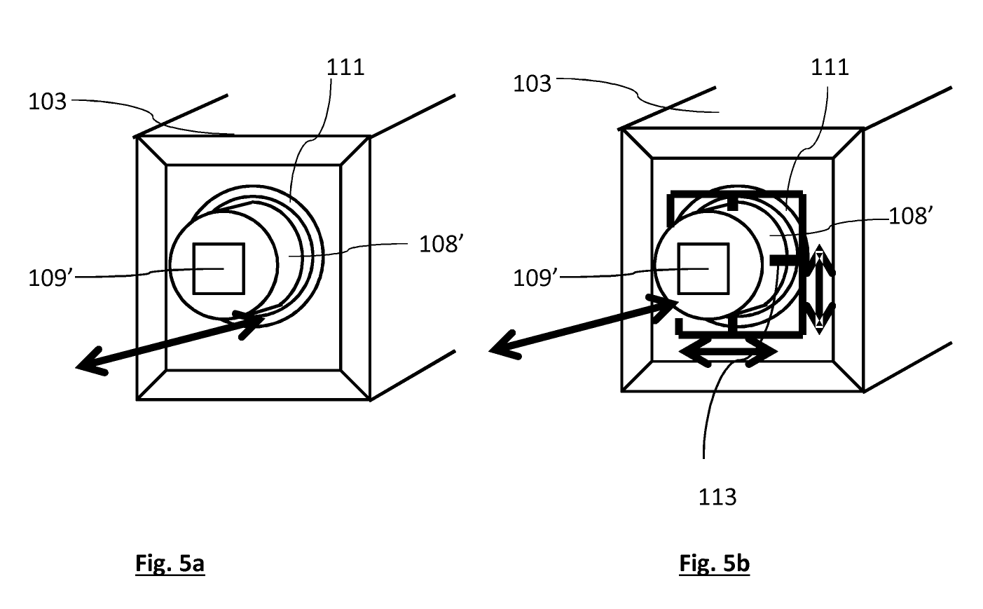

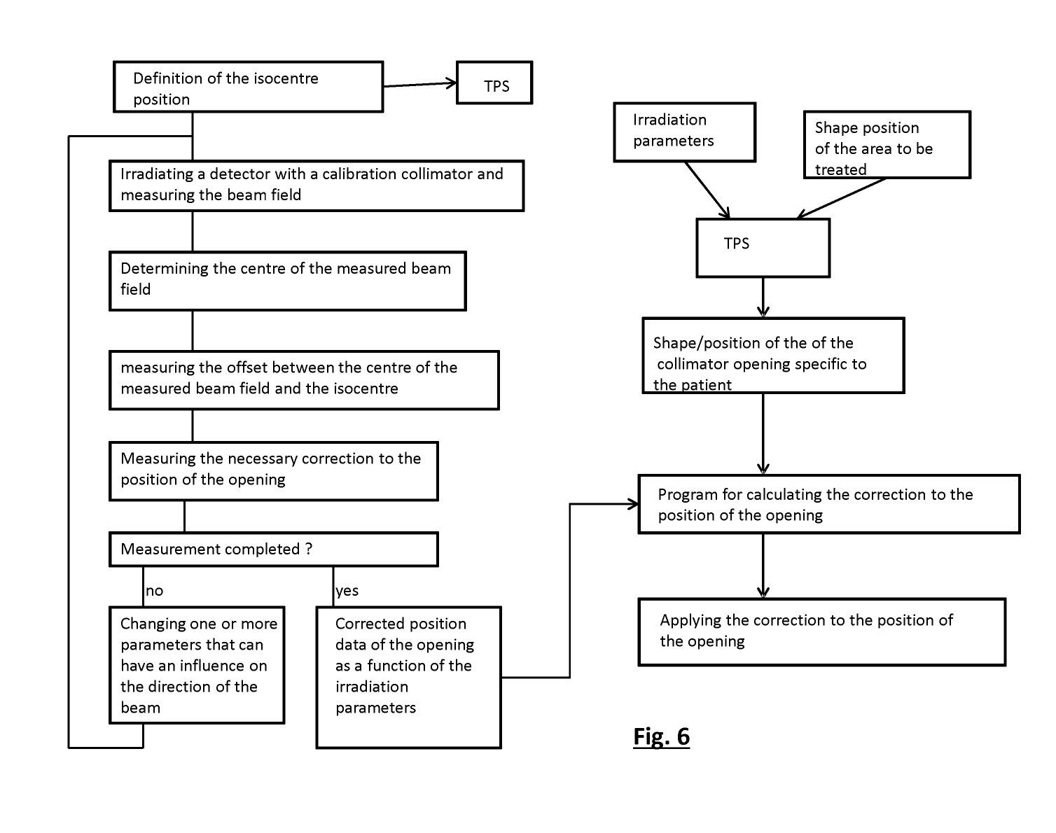

FIG. 6 lays out the various steps and sub-steps according to an embodiment of the method. The isocentre of the rotatable gantry is determined and the data on position of the isocentre are introduced into the treatment planning system. Preferably, the step i) forms part of a calibration step. In this calibration step, a calibration collimator is positioned on the irradiation unit (such an example is shown in FIGS. 5a and 5b). This calibration collimator comprises an opening with a simple shape, for example a circle or a square. A detector is positioned in a precise manner with respect to the isocentre and is irradiated by the irradiation unit using said calibration collimator according to a first parameter that can have an influence on the deviation of the beam or several combined predetermined first parameters that can have an influence on the deviation of the beam, such as for example the angle of rotation of the rotatable gantry and/or the extension of the telescopic support. The beam field projected onto the detector is measured and the centre of the beam field is determined. Subsequently, the offset between the centre of the measured beam field and the position of the isocentre is measured. A program then calculates the correction needed to be applied to the opening of the collimator.

Preferably, calibrated adjustment screws are provided in order to vary the position of the collimator and to allow the correction calculated for the position of the opening of the collimator to be verified. When the measurement and, potentially, the verification of the correction applied are carried out, the parameter that can have an influence on the deviation of the beam or the combined predetermined parameters that can have an influence on the deviation of the beam are modified and several new sequences of beam field measurement, of determination of the centre of the beam field, of measurement of offset between the centre of the beam field and the isocentre, of calculation of the correction to be applied to the position of the opening of the collimator, and of modification of parameters that can have an influence on the deviation of the beam, are carried out. Corrected position data for the opening as a function of each of the predetermined parameters that can have an influence on the deviation of the beam are stored by the program for calculation of the correction needed to be applied to the opening of the collimator.

Preferably, on the basis of the measurements carried out, a calibration curve is established showing the deviation of the beam with respect to the isocentre as a function of one or more parameters that can have an influence on the deviation of the beam such as defined hereinabove. Preferably, this step is carried out for several angles of rotation of the rotatable gantry and for several extensions of the telescopic support.

Prior to treating the patient, the irradiation parameters and data on position and on shape of the area to be treated obtained by imaging of the patient are introduced into the treatment planning system which calculates a treatment plan notably defining, for irradiating the area to be treated:
one or more angles of rotation of the rotatable gantry;
the shape and the position of the opening of the collimator designed for irradiating the area of the patient to be treated, the shape of the compensator designed for irradiating the area of the patient to be treated when the delivery mode of the beam requires one;
the extension of the telescopic support and
the energy of the beam.

The data on shape and position in the 2D plane of the opening of the collimator such as calculated by the treatment planning system are transferred to the program for calculating the correction needed to be applied to the opening of the collimator. The program calculates data on position and, where appropriate, corrected shape of the opening in the collimator designed to allow the irradiation of the area of the patient to be treated on the basis of the data supplied by the treatment plan and of the corrected position data for the opening as a function of each of the predetermined parameters that can have an influence on the deviation of the beam such as calculated during the calibration step. If the pre-determined parameter is the gantry angle, then the program calculates the correction based on the gantry angle that is obtained from the treatment planning. In other words, the program receives as an input a value of the predetermined parameter, the value corresponding to the value defined in the treatment plan. For example the value of a gantry angle for performing a treatment.

These corrected position data for the opening of the collimator designed for irradiating the area of the patient to be treated are transferred to a device capable of forming an opening in a collimator or to a device capable of moving the opening of a collimator.

This method therefore compensates the deviation of the beam with respect to the isocentre by shifting the position and possibly by adapting the shape of the opening of the collimator such as provided by the treatment plan to a corrected position and shape, rather than moving the patient for each new angle of irradiation. The need for carrying out imaging sessions in order to re-centre the area to be treated onto a new isocentre whose position is dependent on the angle of irradiation or on other parameters such as described hereinabove is thus avoided.

According to a first embodiment of the method, when the collimator is a multi-leaf collimator, the step for application of said correction to said opening comprises a step for displacement of said leaves in such a manner as to position said opening of said collimator taking into account the treatment plan and said correction to be applied. The shape of the opening therefore preferably remains identical to that provided by the treatment planning system, but its position is shifted with respect to the position provided by said treatment planning system. The multi-leaf collimator can then be associated with a compensator whose hollow part is aligned with the opening.

According to a second embodiment of the method, when the irradiation unit comprises a device capable of moving a collimator in a plane perpendicular to the axis of the delivered beam, said correction comprises a step for moving said collimator by means of this device, so as to apply the calculated correction. The collimator may be associated with a compensator before or after the application of said correction, the hollow part of the compensator being aligned with the opening of the collimator.

Preferably, the device capable of moving the collimator in a plane perpendicular to the axis of the delivered beam comprises position adjustment screws. This moving device can be motorized or manual and comprise a means for controlling the position of the collimator.

According to a third embodiment of the method, the step for application of said correction comprises a step for fabrication of a collimator whose opening shape and position are based on the shape and the position such as defined by the treatment plan and on the calculation of said correction to be applied to the position of the opening. The collimator may be associated with a compensator, the hollow part of the compensator being aligned with the opening of the collimator.

When the collimator is a block with an opening, it is advantageous to check the quality of fabrication of this collimator. For this purpose, a scale plan of the shape of the collimator, of the shape and the position of its opening such as predetermined on the basis of the treatment plan and, potentially, on the basis of the calculation of the correction to be applied, can be printed, so as to be laid onto the collimator and to verify that the position and the shape of the opening in the collimator are correct.

According to a second aspect, the invention relates to a program for a hadron-therapy installation such as described hereinabove. The program comprises a calculation algorithm allowing the calculation of a correction to be applied to the position of an opening of a collimator such as defined by a treatment plan, for compensating the deviation of the beam with respect to the isocentre as a function of parameters that can have an influence on the direction of the beam.

Said program calculates said correction to be applied on the basis:
of data from a treatment plan defining the shape and the position of said opening in the collimator; and
of a determination of the deviation of the beam with respect to said isocentre as a function of parameters that can have an influence on the direction of the beam.

Preferably, the program for calculation of the correction to be applied is based on the determination of the deviation of the beam with respect to the isocentre as a function of one or more parameters selected from amongst:
the angle of rotation of the rotatable gantry;
the extension of the telescopic support 111 supporting the accessories 108, 110;
the weight of the accessories 108, 110;
the type of telescopic support used and adapted to the weight of the accessories;
the mode of treatment (double scattering, Uniform scanning, Pencil beam scanning or Single scattering);
the irradiation parameters (position, presence of beam modulator, of beam widener, etc.).

Preferably, the program creates corrected position data for the opening in the collimator designed for irradiating the area of the patient to be treated, on the basis of the data supplied by the treatment plan and on the basis of the calculation of the correction to be applied to the position of the opening.

According to a first embodiment of the program, the corrected position data for the opening of the collimator are transmitted to a device capable of forming the opening of the collimator.

In the case where a multi-leaf collimator is used, these corrected position data for the opening of the collimator are transferred to a system for controlling a motorization device for the plurality of leaves of said multi-leaf collimator.

In the case where a collimator formed from a block with an opening is used, these corrected position data for the opening of the collimator are transferred to a device for controlling a collimator fabrication device.

In the case of an irradiation unit comprising a device for moving a collimator in a plane perpendicular to the direction of the beam, when this device comprises, for example, a collimator support that may be actuated by a motor, the corrected position data for said opening of the collimator are communicated to a system for controlling the device so as to correct the position of the opening. If this device is manual and comprises, for example, a system of screws associated with a visual alignment mark, these corrected position data for the opening in the collimator are communicated in the form of instructions to the user.

According to a second embodiment of the program, the corrected position data for the opening of the collimator are transmitted to a printer device for printing a plan of said collimator on a 1:1 scale, the plan being designed to verify the correct position of the opening of the collimator.

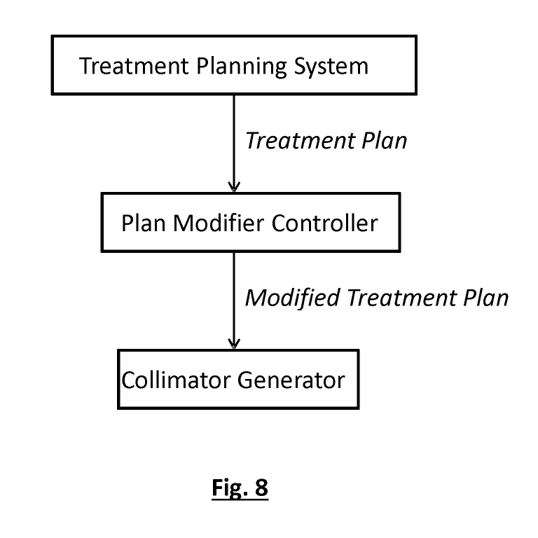

FIG. 8 illustrates a more general view of the workflow followed when applying the method of the invention. This figure also illustrates the main building blocks of a hadron therapy installation according to an embodiment of the invention. A treatment planning system defines a treatment plan for performing a treatment at a given treatment angle. This treatment plan comprises prescribed data that define a prescribed opening on the collimator. A plan modifier controller then modifies the plan by applying a correction to the prescribed data defining the prescribed opening. This modification or correction is based on a calibration measurement performed prior to the treatment. This calibration measurement measures the deviation of the beam with respect to an isocentre as a function of a parameter, e.g. the gantry angle, allowing to determine the calibration curve or curves described with respect to the method of the invention. After the corrected data for the opening on the collimator are defined, the physical realisation of the collimator can be made. The collimator generator can for example be a milling machine that is milling a opening in a collimator block based on the corrected data defining the corrected opening. In stead of a collimator generator, the corrected data can be sent to collimator positioner, i.e. the collimator already provided with an opening of the prescribed shape can be moved to a corrected position.

For the purpose of calculating the correction to be applied to the prescribed data defining the prescribed opening, the plan modifier controller will need to have access to the calibration data defining the deviation of the beam from isocentre as a function of the parameter. For this purpose the hadron-therapy installation (100) comprises a storage medium configured for storing data related to the deviation of the beam (104) with respect to an isocentre (107) as a function of one or more irradiation parameters of the installation that can have an influence on the deviation of the beam. These ‘data related to the deviation’ can the calibration curves as such or data allowing to determine said curves.

The plan modifier controller can either be a controller independent from the treatment planning system or it can be a controller that is integrated in the treatment planning system.

As stated above, the ‘isocentre of a structure supporting said irradiation unit’ cited in appended claim 1 is a point with a fixed position measured with respect to the structure itself (e.g. rotatable gantry or fixed beam), or to the space wherein the structure is located, i.e. with respect to the treatment room. In EP1759733, the ‘isocentre’ is the origin of a reference coordinate system fixed in the positioning system of the irradiation unit. Said positioning system positions the unit with respect to the structure (e.g. a rotatable gantry). This means that for different positions of the irradiation unit, for example for different rotation angles of a rotatable gantry, the ‘isocentre’ of D1 is not located in the same spatial point of the treatment room, due to the deviation that depends on the rotation angle. It is the latter deviation, i.e. a deviation with respect to point defined in a coordination system that is not attached to the irradiation unit, that is corrected by the method and program of the invention.

It is to be noted that the method steps i to iv of appended claim 1 are generally not performed in sequence (although this is not excluded from the claim scope). As stated above, step i takes place in the form of a calibration procedure, performed prior to any specific treatment. Step i also does not require the shape and position of a collimator opening provided by a treatment plan, but can be performed with the use of a calibration collimator with a predefined opening, e.g. a square or circular shaped opening. One or more calibration curves are the result of step i, e.g. a curve establishing the deviation with respect to the isocentre for a plurality of gantry angles and another curve showing the deviation for a plurality of telescopic support extension positions.

Step ii (obtaining a treatment plan) is performed for every specific treatment, as every treatment requires a specific treatment plan, defining the position and shape of the collimator opening, and the installation parameters such as the gantry angle and the telescopic support extension that are to be applied in that treatment.

Step iii can be performed for every treatment plan, i.e. based on the prescribed treatment plan parameters, the beam deviation is derived from the calibration curve(s), and a correction is calculated only for the values of the parameters described in the treatment plan. These corrections are then to be applied to the prescribed collimator opening for compensating the deviation.

Alternatively, said corrections are calculated beforehand, for every value of the installation parameters. In other words, the calibration curves for the deviation as a function of the parameters are translated to calibration curves for the correction (e.g. delta-X and delta-Y corrections) with respect to a reference position, as a function of said parameters, irrespective of a prescribed collimator shape and position. In that case, the corrections to be applied to the position of a prescribed opening can be determined directly from said curves.

Whenever a ‘block’ collimator is referred to in the present description, this can be either a solid block or a block consisting of a plurality of layers of solid material.

1. A method for compensating the deviation of an energetic beam (104) of hadrons delivered by an irradiation unit (103) of a hadron-therapy installation (100), with respect to an isocentre (107) of a structure supporting said irradiation unit (103), said irradiation unit (103) being configured for receiving a collimator (108) having an opening (109) for the passage of said beam (104), said compensation method comprising the steps of:
i. determining the deviation of the beam (104) with respect to said isocentre (107) as a function of a parameter of said installation that can have an influence on the deviation of said beam, thereby obtaining a calibration curve of said deviation as a function of a plurality of values of said parameter;
ii. obtaining a treatment plan from a treatment planning system, said plan defining a prescribed shape and position of said collimator opening (109) for performing a treatment,
iii. calculating a correction to be applied to said prescribed position of said opening (109) for compensating the deviation of said beam (104) with respect to said isocentre (107), said correction being calculated on the basis of said calibration curve;
iv. application of said correction to said prescribed position of said opening (109).

2. The method according to claim 1, wherein said structure is a rotatable gantry, and wherein said parameter that can have an influence on the deviation of said beam (104) is the angle of rotation of said rotatable gantry (105).

3. The method according to claim 2, wherein said irradiation unit (103) comprises a telescopic support (111) for the positioning of accessories such as said collimator (108) associated or not with a compensator (110), the method further comprising determining the deviation of the beam (104) with respect to said isocentre (107) as a function of at least a second parameter that can have an influence on the deviation of said beam, said second parameter being selected from:
the extension of the telescopic support (111) supporting the accessories (108, 110);
the weight of the accessories (108, 110);
the type of telescopic support (111) used and adapted to the weight of the accessories;
the mode of treatment (double scattering, Uniform scanning, Pencil beam scanning or Single scattering);
the irradiation parameters (position, presence of beam modulator, of beam widener, etc.).

4. The method according to claim 1, wherein said structure is a fixed beam structure, and wherein said irradiation unit (103) comprises a telescopic support (111) for the positioning of accessories such as said collimator (108) associated or not with a compensator (110), and wherein said parameter is selected from:
the extension of the telescopic support (111) supporting the accessories (108, 110);
the weight of the accessories (108, 110);
the type of telescopic support (111) used and adapted to the weight of the accessories;
the mode of treatment (double scattering, Uniform scanning, Pencil beam scanning or Single scattering);
the irradiation parameters (position, presence of beam modulator, of beam widener, etc.).

5. The method according to claim 1, wherein said step of determining the deviation of the beam (104) with respect to said isocentre (107) as a function of a parameter is carried out according to the following sub-steps:
a) at a first value of said parameter, irradiation of a detector using a calibration collimator (108′), said detector (130) being positioned with respect to the isocentre;
b) measurement of the beam field (104) by means of said detector (130);
c) determination of the centre of the beam field (104);
d) measurement of the offset between the centre of the measured beam field and the isocentre;
e) changing the value of said parameter and repeating steps a) to d).

6. The method according to claim 1, wherein said collimator (108) is a multi-leaf collimator, said step for application of said correction to the prescribed position of said opening (109) comprises a step for displacement of said leaves.

7. The method according to claim 1, wherein said collimator (108) is a block comprising an opening (109), and when said collimator is fixed onto a device (112) capable of moving it in a plane perpendicular to the direction of the beam, said step for application of said correction to said prescribed position of said opening (109) comprises a step for displacement of said collimator (108) by means of said device (112).

8. The method according to claim 1, wherein said step for application of said correction to said prescribed position of said opening (109) comprises a step for fabrication of a collimator (108) whose opening (109) shape and position is based on said treatment plan and on the calculation of said correction to be applied.

9. The method according to claim 1, wherein said collimator (108) is associated with a compensator (110) comprising a part whose shape is predetermined on the basis of said treatment plan and aligned with the corrected position of the opening (109) of said collimator (108).

10. The method according to claim 1, further comprising the step of calculating a correction to be applied to said prescribed shape of said opening (109) for compensating the deviation of said beam (104) with respect to said isocentre (107), said correction being calculated on the basis of said calibration curve.

11. A program comprising an algorithm for calculation of a correction to be applied to the position of an opening of a collimator of an irradiation unit of a hadron-therapy installation comprising a structure supporting said irradiation unit, the shape and the position of said opening being prescribed by a treatment plan, said correction to be applied being capable of compensating the deviation of the beam produced by said hadron-therapy installation, with respect to the isocentre of said structure, as a function of a parameter that can have an influence on the direction of the beam, wherein said correction is calculated on the basis of a calibration curve of said deviation as a function of a plurality of values of said parameter.

12. The program according to claim 11, wherein said structure is a rotatable gantry and wherein said parameter that can have an influence on the direction of said beam is the angle of rotation of said rotatable gantry.

13. The program according to claim 12, wherein said irradiation unit (103) comprises a telescopic support (111) for the positioning of accessories (108, 110) such as said collimator (108) associated or not with a compensator (110), the program being characterized in that said correction to be applied is capable of compensating the deviation of the beam produced by said hadron-therapy installation with respect to the isocentre of said gantry, as a function of one or more second parameter(s) selected from:
the extension of the telescopic support (111) supporting the accessories (108, 110);
the weight of the accessories (108, 110);
the type of telescopic support used and adapted to the weight of the accessories;
the mode of treatment (double scattering, Uniform scanning, Pencil beam scanning or Single scattering);
the irradiation parameters (position, presence of beam modulator, of beam widener, etc.).

14. The program according to claim 11, wherein said structure is a fixed beam structure, and wherein said irradiation unit (103) comprises a telescopic support (111) for the positioning of accessories such as said collimator (108) associated or not with a compensator (110), and wherein said parameter is selected from:
the extension of the telescopic support (111) supporting the accessories (108, 110);
the weight of the accessories (108, 110);
the type of telescopic support (111) used and adapted to the weight of the accessories;
the mode of treatment (double scattering, Uniform scanning, Pencil beam scanning or Single scattering);
the irradiation parameters (position, presence of beam modulator, of beam widener, etc.).

15. The program according to claim 11, wherein the program is capable of establishing said calibration curve.

16. The program according to claim 11, wherein the program creates corrected position data for the opening (109) of a collimator (108),
on the basis of data coming from a treatment plan prescribing the shape and the position of said opening in the collimator for a given isocentre; and
on the basis of the calculation of the correction to be applied to the position of said opening.

17. The program according to claim 16, wherein said corrected position data for the opening of the collimator (108) are transmitted to a device (112) capable of forming said opening.

18. The program according to claim 17, wherein said device (112) capable of forming said opening (109) is a control system for motorization of a plurality of leaves of a multi-leaf collimator.

19. The program according to claim 17, wherein said device (112) capable of forming said opening (109) is a control system for a collimator fabrication device.

20. The program according to claim 16, wherein said corrected position data for the opening (109) of said collimator (108) are transmitted to a printer device for printing a plan of said collimator on a 1:1 scale, said plan being designed to verify the correct position of said collimator.

21. The program according to claim 16, wherein said corrected position data are transmitted to a device (112) capable of moving said collimator (108).

22. A hadron-therapy installation (100) for delivering a hadron beam for hadron therapy, said hadron-therapy installation comprising:
an irradiation unit (103) for delivering said beam, said irradiation unit (103) being configured for receiving a collimator (108) having an opening (109) for the passage of the beam,
a structure for (105) supporting said irradiation unit (103),
a treatment planning system for providing a treatment plan, said treatment plan comprising prescribed collimator data defining the shape of the opening and the position of the opening of the collimator,
a storage medium configured for storing data related to the deviation of the beam (104) with respect to an isocentre (107) as a function of one or more irradiation parameters of said installation that can have an influence on the deviation of said beam,
a plan modifier controller adapted to modify said prescribed collimator data of said treatment plan by defining a correction to the position of said opening of said collimator, wherein said defining a correction is based on said data related to the deviation of the beam (104).

23. The hadron therapy installation according to claim 22, wherein said structure is a rotatable gantry, and wherein said parameters that can have an influence on the deviation of said beam (104) comprise at least the angle of rotation of said rotatable gantry (105).

24. The hadron therapy installation according to claim 22, wherein said structure is a fixed beam structure and wherein said irradiation unit (103) comprises a telescopic support (111) for the positioning of accessories such as said collimator (108) associated or not with a compensator (110), and wherein said one or more parameters are selected from:
the extension of the telescopic support (111) supporting the accessories (108, 110);
the weight of the accessories (108, 110);
the type of telescopic support (111) used and adapted to the weight of the accessories;
the mode of treatment (double scattering, Uniform scanning, Pencil beam scanning or Single scattering);
the irradiation parameters (position, presence of beam modulator, of beam widener, etc.).
